(19) United States
(12) Patent Application Publication
(10) Pub. No.: US 2007/0142779 A1
(43) Pub. Date: Jun. 21, 2007

(54) Catheter for guidewire placement

(75) Inventors: Patrick Duane, Galway (IE); Terry Guinan, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/314,942

(22) Filed: Dec. 20, 2005

(51) Int. Cl. A61M 29/00; A61M 5/178; A61M 31/00; A61M 37/00

(52) U.S. Cl. 604/164.09; 604/103.04

(57) ABSTRACT

A catheter that has a proximal portion, a transition portion, a distal portion, and a guide member. The guide member is slideably coupled to the proximal portion and provides access to a guidewire lumen within the catheter through a guideway that extends from an outer surface of the proximal portion to the guidewire lumen. The distal portion may include a branch lumen that exits the catheter through the wall of the distal portion so that a guidewire may be guided through a particularly tortuous vessel.

### FIELD OF THE INVENTION

The present invention relates to medical catheters. More specifically, the invention relates to a catheter used for the placement of a guidewire that includes a guide member, which facilitates control over the guidewire independent of the catheter.

### BACKGROUND OF THE INVENTION

Cardiovascular disease, including atherosclerosis, is a leading cause of death in the U.S. As a result, many procedures have been developed to treat and diagnose various conditions that arise from cardiovascular disease. Such procedures include percutaneous transluminal coronary angioplasty, commonly referred to as “angioplasty” or “PTCA”, implantation of vascular prosthesis or stents, delivery of therapeutic substances (such as anti-vaso-occlusion agents or tumor treatment drugs), delivery of radiopaque agents for radiographic viewing, and making intravascular pressure measurements.

The objective in angioplasty is to enlarge the lumen of the affected coronary artery by radial hydraulic expansion. The procedure is accomplished by locating a guidewire in the narrowed region of the coronary artery. The balloon of a balloon catheter is then positioned within the narrowed region of the coronary artery by advancing the balloon catheter over the guidewire. The balloon is subsequently inflated and the radial expansion of the balloon causes soft, fatty plaque deposits to be flattened and hardened deposits to be cracked and split. As a result, the lumen is enlarged.

One or multiple dilations may be necessary to effectively enlarge the arterial lumen. In cases where successive dilations are required, they may be applied using a series of balloon catheters having balloons with increasingly larger diameters. Additionally an intravascular prosthesis, or stent, may be implanted inside the artery at the site of the lesion to help prevent arterial closure and/or restenosis or to reinforce the vessel wall after dilation.

Conventional catheter shafts typically include a proximal portion, a transition portion and a distal portion that terminates at a flexible tip. Generally, the proximal portion is relatively rigid to allow for increased pushability and includes a guidewire lumen extending throughout its length. In contrast, the distal portion is generally a flexible polyethylene sleeve with a flexible polyethylene tube disposed concentrically within the sleeve that extends the guidewire lumen from a distal end of the proximal portion to the distal tip of the catheter. Typically, the distal portion extends for a length on the order of 25 centimeters which allows the catheter to curve through particularly tortuous vessels over a guidewire. The transition portion provides a gradual transition in stiffness between the relatively stiff proximal portion and the flexible distal portion. The transition in stiffness reduces the tendency of the catheter shaft to collapse, buckle or kink, particularly, where the rigid proximal portion and the flexible distal portion meet.

Two types of catheters that are commonly used with a guidewire are referred to as over-the-wire (OTW) catheters and rapid exchange (RX) catheters. A third type of catheter with preferred features of both OTW and RX catheters, is sold by Medtronic Vascular, Inc. of Santa Rosa, Calif. under the trademarks MULTI-EXCHANGE, ZIPPER MX, ZIPPER, and/or MX, (hereinafter referred to as the “MX catheter”). All three types of catheters are discussed below in greater detail.

An OTW catheter's guidewire lumen runs the entire length of the catheter. Thus, the entire length of an OTW catheter is tracked over a guidewire when the catheter is positioned during a procedure. If a catheter exchange is required while using a standard OTW catheter, the clinician must add an extension wire onto the proximal end of the guidewire to maintain control over the guidewire. The indwelling catheter may then be slid off of the extended guidewire. A subsequent catheter can then be loaded onto the guidewire and tracked to the treatment site. A major disadvantage of OTW catheters is that multiple operators are required to hold the extended guidewire in place to maintain its sterility while the catheter is exchanged.

In contrast, a RX catheter has a guidewire lumen that has a relatively short length extending through only a portion of the catheter near the distal end. In other words, the guidewire is located outside of the catheter except for a comparatively short segment at the distal end of the catheter. Thus, when using a RX catheter, only a distal portion of the catheter is tracked over the guidewire. During catheter exchanges, rapid exchange catheters avoid the need for multiple operators and as a result are often referred to as a “single operator” catheter. Since the majority of the guidewire is exposed, the guidewire can be held in place without requiring a guidewire extension while the catheter is retracted. Once the original RX catheter is removed, another catheter may be threaded onto the guidewire and tracked to the treatment site.

Although the RX catheter may provide the advantages discussed above, it presents several disadvantages. First, without a full-length guidewire lumen, the proximal shaft of a RX catheter cannot rely on the guidewire for stiffness, or conversely the guidewire cannot rely on the catheter for added stiffness. The coaxial relationship between a guidewire and an OTW catheter provides desirable transmission of force along the catheter length and aids a clinician when advancing the catheter and guidewire through tight stenoses and/or tortuous blood vessels. Accordingly, even if an OTW catheter begins to kink slightly when the catheter is advanced through a tight stenosis, the coaxial guidewire limits the kinking of the catheter and most of the pushing force is still transmitted to the distal tip of the catheter and guidewire combination. Since the RX catheter does not allow such a coaxial relationship with a guidewire, the pushing force is not transmitted as efficiently.

A second disadvantage is that guidewire exchanges with an indwelling RX catheter are not possible. The proximal guidewire port of a RX catheter is located remotely within the patient on an indwelling RX catheter. As a result, if the guidewire becomes damaged, if a different guidewire design becomes desirable, or if the guidewire is unintentionally withdrawn, it is not feasible to exchange or reposition the guidewire without removing the RX catheter.

An additional disadvantage of RX catheter systems is that they can be difficult to seal against blood loss. The RX catheter and the guide wire extend from the guiding catheter side-by-side, making it awkward to seal. The sealing, or “anti-backbleed” function is typically accomplished with a “Tuohy-Borst” fitting that has a manually adjustable gasket with a round center hole. The adjustable gasket does not conform well to the side-by-side arrangement of a RX catheter and guidewire.

Another disadvantage of RX catheters is the lack of a full-length guidewire lumen. The absence of a full-length guidewire lumen deprives the clinician of an additional lumen that may be used for other purposes. For example, the extra lumen could be utilized for pressure measurement, injection of contrast dye, or infusing a drug.

The MX catheter is generally capable of both fast and simple guidewire and catheter exchange thereby addressing some of the deficiencies of both RX and OTW catheters. The MX catheter is disclosed in U.S. Pat. No. 4,988,356 to Crittenden et al, U.S. Pat. No. 6,800,065 to Duane et al, U.S. Pat. No. 6,893,417 to Gribbons et al, and U.S. Pat. No. 6,905,477 to McDonnell et al., and also in U.S. Patent Application Publication 2004-0059369 A1 published Mar. 25, 2004, and U.S. Patent Application Publication 2004-0260329 A1 published Dec. 23, 2004, all of which are incorporated by reference in their entirety.

The MX catheter includes a catheter shaft having a guideway that extends longitudinally along the catheter shaft and radially from a guidewire lumen to an outer surface of a catheter shaft, and a guide member. The guide member is slideably coupled to the catheter shaft and cooperates with the guideway, such that a guidewire may extend transversely into or out of the guidewire lumen at any location along the length of the guideway. By moving the shaft with respect to the guide member, the effective over-the-wire length of the MX catheter is adjustable. As a result of the variable over-the-wire length, catheter exchanges may be performed without requiring extension wires and guidewire exchanges are possible.

The OTW, RX and MX catheters depend upon a guidewire to guide them to the proper location. As a result, their use may be limited by the ability to properly place a guidewire.

One source of complexity in positioning catheters may be stenoses, or other blockages in a patient's vessel that inhibit the travel of a guidewire. Sometimes the stiffness of a particular guidewire is not sufficient to breach a blockage. In those cases, a clinician may desire to increase the stiffness of the guidewire without being required to perform a guidewire exchange or may desire to have a tool specifically designed to assist in breaching a stenosis. Another source of difficulties in the placement of guidewires is the uniqueness of each patient's vasculature. A patient's coronary arteries may be irregularly shaped, highly tortuous, and/or very narrow. However, guidewires are not always capable of navigating some tortuous vessels. For example, where the guidewire must be directed through a sharp turn to reach the treatment site. In those cases a clinician may desire a tool that would lead a guidewire through a particularly sharp turn. Therefore, a need exists to provide a catheter that aids in the placement of a guidewire at a treatment site and allows for efficient guidewire and catheter exchanges.

### BRIEF SUMMARY OF THE INVENTION

An embodiment of the present invention is a catheter that aids in the placement of a guidewire. The catheter includes a tubular catheter shaft that has a distal tip, which may be cut flush or have a profiled taper, a guidewire lumen extending longitudinally through the catheter shaft, a guideway that extends from the outer surface of the catheter shaft to the guidewire lumen, and a guide member slidably coupled to the catheter shaft. In one embodiment, the distal tip is of the same material as the remainder of the catheter shaft but may have a greater stiffness. Loading the catheter onto a guidewire allows a clinician to easily increase the stiffness of the guidewire. The increased stiffness in addition to the configuration of the distal tip allow a clinician to more easily breach stenoses.

In another embodiment of the present invention, the catheter includes a tubular catheter shaft, a guidewire lumen extending longitudinally through the catheter shaft, a guideway that extends from the outer surface of the catheter shaft to the guidewire lumen, a guide member slidably coupled to the catheter shaft, and a branch lumen. The branch lumen extends longitudinally through a portion of the catheter shaft and exits through a side wall of the catheter shaft at an angle up to 90° and allows a clinician to advance a guidewire through a particularly tortuous vessel.

In another embodiment of the present invention, the catheter includes a tubular catheter shaft, a guidewire lumen extending longitudinally through the catheter shaft that has a diameter that reduces along a length of the catheter from a diameter that is significantly larger than the guidewire in a proximal portion to a diameter that approaches the guidewire diameter in a distal portion. A guideway extends from an outer surface of the catheter shaft to the guidewire lumen, and a guide member is slidably coupled to the catheter shaft.

In another embodiment of the present invention, the catheter for guidewire placement includes a catheter shaft having a proximal portion and a distal portion, wherein the distal shaft portion includes a necked region that transitions the distal shaft from a first outer diameter to a reduced second outer diameter. A guidewire lumen and an auxiliary lumen extending longitudinally through the catheter shaft in a side-by-side arrangement in the proximal shaft, wherein the auxiliary lumen is used to accommodate drug or dye infusion. A guideway extends longitudinally along the length of the proximal portion and radially from the guidewire lumen to an outer surface of the proximal portion. A guide member is slideably coupled to the catheter shaft and is configured to provide access to the guidewire lumen via the longitudinal guideway. In a further embodiment, the first outer diameter of the distal shaft portion is 2.7 F and the second outer diameter is 2.5 F to fit within tightly stenosed occlusions.

Additionally, an embodiment of the present invention provides for a method of using a catheter for placing a guidewire. The method includes the steps of providing a catheter, and a guidewire, backloading the guidewire into the catheter by inserting a proximal end of the guidewire into a branch lumen exit of the catheter and sliding the guidewire further proximal into the branch lumen until the guidewire is fully inserted into the branch lumen. The method further includes advancing the catheter so the branch lumen exit is aligned with a tortuous vessel, and advancing the guidewire distally through the branch lumen so that a distal tip of the guidewire exits the branch lumen exit and enters the tortuous vessel.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art based on the teachings contained herein.

### BRIEF DESCRIPTION OF THE DRAWINGS

These and other features, aspects and advantages of the present invention will become better understood with reference to the following description, appended claims, and accompanying drawings. The drawings are not to scale.

FIG. 1 is an isometric view of a catheter according to one aspect of the present invention.

FIG. 2 is a side view of the catheter of FIG. 1.

FIG. 3 is a cross-sectional view of a proximal portion of the catheter of FIG. 2 taken along line A-A.

FIGS. 4A-4C illustrate various embodiments of stiffening features integrated into the wire exchange catheter of FIG. 2 shown in a cross-sectional view taken along line A-A.

FIGS. 5A and 5B illustrate two embodiments of transition portions of the stiffening features of FIGS. 4B and 4C.

FIG. 6 is a partial sectional top view of a distal portion of the catheter of FIG. 1.

FIG. 7 is a cross-sectional view of the catheter of FIG. 2 taken along line B-B.

FIG. 8 is a partial sectional view of an embodiment of the catheter that includes a branch lumen.

FIGS. 9A-9C illustrate various embodiments of the catheter of FIG. 8 shown as a cross-sectional view taken along line C-C.

FIG. 10 is a side view of an alternative embodiment of the catheter of FIG. 8.

FIG. 11 is an isometric view of one embodiment of the guide member of FIG. 1.

FIG. 12 is a cross-sectional view of the guide member of FIG. 11 taken on plane D.

FIG. 13 is a cross-sectional view of the guide member of FIG. 11 taken on plane E.

FIG. 14 is an isometric view of an alternative embodiment of the guide member of FIG. 1.

FIG. 15 is an isometric view of an outer tubular member of the guide member of FIG. 14.

FIG. 16 is side elevational view of an inner body of the guide member of FIG. 14.

FIG. 17 is a cross-sectional view of the inner body of FIG. 16 taken on plane F.

FIG. 18 is an isometric view of a further alternative embodiment of the guide member of FIG. 1.

FIG. 19 is a cross-sectional view of the guide member of FIG. 18 taken on plane G.

FIG. 20 is a cross-sectional view of the guide member of FIG. 18 taken on plane H.

FIG. 21 is a side view of a portion of a catheter according to an embodiment of the present invention.

FIG. 22 is a side view of a portion of a catheter according to an embodiment of the present invention.

FIG. 23 is a perspective view of an alternative embodiment of a catheter according to the present invention.

FIGS. 24, 24A and 25 are cross-sectional views of various embodiments of a distal portion of the catheter shown in FIG. 23.

### DETAILED DESCRIPTION OF THE INVENTION

The present invention is now described with reference to the figures where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

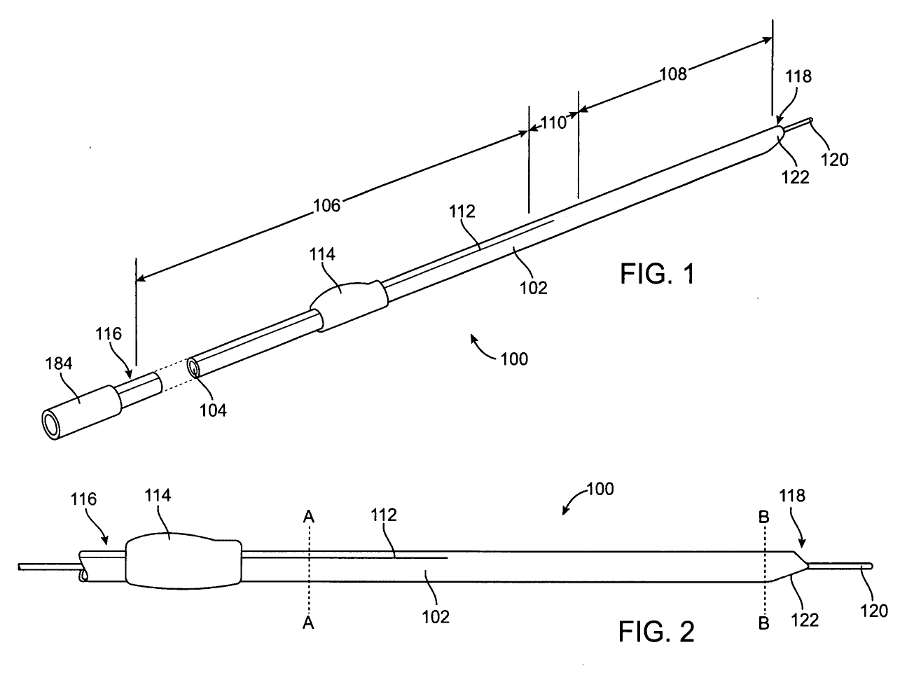

An embodiment of the catheter of the present invention is shown in FIGS. 1 and 2 and indicated generally by reference numeral 100 that includes the features of an MX catheter and a distal tip 122 specifically configured to breach stenoses. Catheter 100 has a catheter shaft 102 having a proximal portion 106 on which a guide member 114 is slideably coupled, a transition portion 110, and a distal portion 108. A guidewire 120 is shown extending out of the distal tip 122 of catheter shaft 102. Guidewire 120 is slideably received within a guidewire lumen 104.

Guide member 114 slides longitudinally along proximal portion 106 and allows a clinician to access guidewire lumen 104 through a guideway 112. Guideway 112 extends longitudinally along a proximal portion 106 substantially from proximal end 116 to transition portion 110, and radially from guidewire lumen 104 to an outer surface of proximal portion 106. It shall be appreciated that guide member 114 generally allows the clinician to independently control guidewire 120 and catheter shaft 102 while guide member 114 is located at any point along the length of guideway 112 of proximal portion 106. In operation, spreading guideway 112 provides a thoroughfare for direct access to guidewire lumen 104.

Catheter shaft 102 is an elongate, flexible, tubular shaft which may be formed from polymeric materials, including high-density polyethylene, polyimide, polyamides, polyolefins, PEBAX® polyethylene block amide copolymer and various other polymeric materials suitable for use in medical devices. Preferably, catheter shaft 102 is made from high-density polyethylene due to its low friction characteristics. Generally, the portions may be integrated into one body, such as through one extrusion process, or catheter shaft 102 may be constructed by coupling individual portions. As shown in the illustrated embodiment, catheter shaft 102 may be circular in shape, but it is not restricted to that configuration.

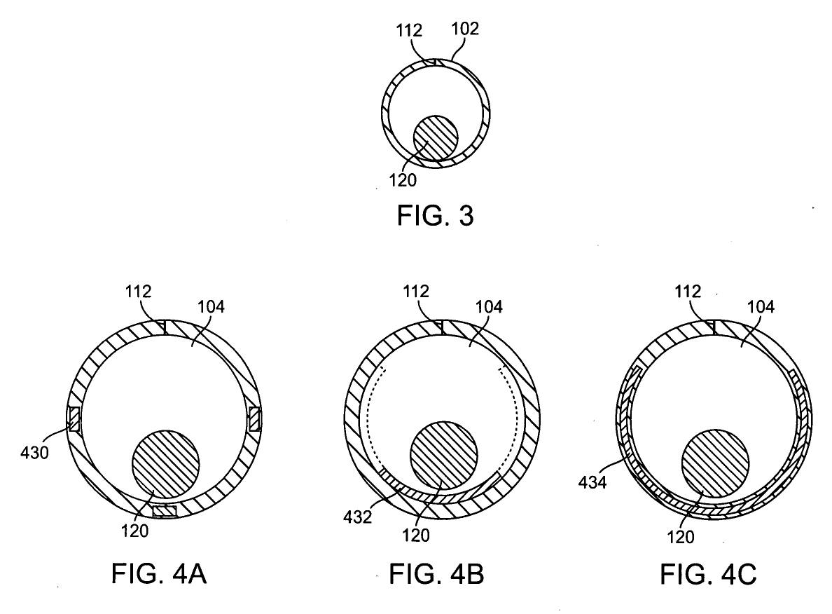

Proximal shaft portion 106 is the longest portion of catheter shaft 102 as compared to transition portion 110 and distal portion 108. As shown in FIG. 3, proximal portion 106 is tubular and has both an inner diameter and an outer diameter that are generally constant. The inner diameter of proximal portion 106 is configured to be substantially larger than an outer diameter of guidewire 120. The difference in diameters allows guidewire 120 to freely move through proximal portion 106. In an embodiment, the inner diameter of proximal portion 106 may be up to twice the outer diameter of guidewire 120.

As shown in FIGS. 6 and 7, distal shaft portion 108 includes a single lumen, i.e., guidewire lumen 104, that ends at distal tip 122. Within distal portion 108, a diameter of the distal portion of guidewire lumen 104 is reduced from a maximum diameter at its proximal end adjacent the inner diameter of proximal portion 106 to a minimum diameter that approaches an outer diameter of guidewire 120 at its distal end.

The outer diameter of catheter tip 122 reduces until it approaches the outer diameter of guidewire 120. Catheter tip 122 is tapered so that it may easily traverse tortuous vessels. In addition, the taper may ease the ability to breach stenoses as the catheter is driven through the vasculature system.

Distal tip 122 may be shaped for a particular response. For example, it may be tapered or curved to match the design of the tip of guidewire 120 or it may provide a shallow taper so that it may more easily penetrate a blockage. In an embodiment, distal tip 122 is made stiffer than the remainder of catheter shaft 106.

Reinforcement may be included in or on catheter tip 122 so that catheter tip 122 is resistant to deformation when it is used to breach built up material within a vessel. For example, a metallic insert may be extruded with the catheter shaft.

The stiffness of proximal portion 106 may also be customized. The stiffness of proximal portion 106 may be derived solely from the characteristic stiffness of the material and shape of proximal portion 106. Alternatively, additional stiffening features may be included, as shown in the various embodiments illustrated in FIGS. 4A-C which are various cross-sections along line A-A of FIG. 2. As is apparent from the figures, various embodiments of stiffening features are available that do not impede access to guidewire lumen 104 via guideway 112.

Multiple stiffening wires 430 may be extruded into proximal portion 106 as shown in FIG. 4A. Stiffening wires 430 are shown having rectangular cross-sectional shapes, however stiffening wires 430 may have any cross-sectional shape that provides the desired stiffness.

Alternatively, as shown in FIG. 4B, a stiffening insert 432 may be included within the guidewire lumen 104. Stiffening insert 432 may be lubricated to reduce friction between guidewire 120 and stiffening insert 432 as guidewire 120 slides through guidewire lumen 104. Alternatively, stiffening insert 432 may be constructed from a material that will not restrict guidewire 120 from sliding through guidewire lumen 104.

As shown in FIG. 4C, a stiffening tube 434 may also be extruded into the wall of proximal portion 106. In this embodiment, stiffening tube 434 is a partial tube so that access to guidewire lumen 104 through guideway 112 is not hindered.

The stiffening features may be constructed from metal or polymer and may be formed from wire, rod or plate in a flat, or curved shape. If the stiffening feature is curved, it can be pressed into its curved shape, cut from a hypotube, or extruded into a curved shape. Metal stiffening features may be constructed from stainless steel, titanium, tungsten, nitinol or any other metal known in the art suitable for use in medical devices. If a polymeric material is used, it may be any polymeric material having high rigidity and suitable for use in medical devices.

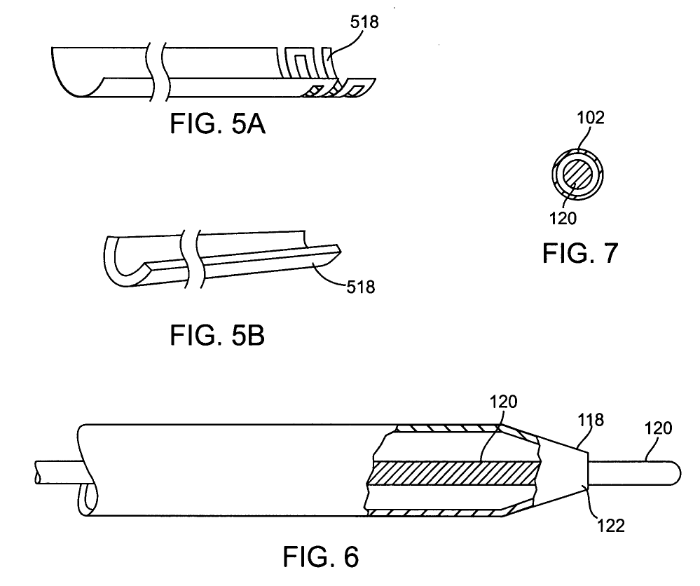

With reference to FIGS. 1 and 2, catheter 100 may include transition section 110 where the stiffness is gradually reduced between the relatively stiff proximal portion 106 and the relatively flexible distal portion 108. FIGS. 5A-5B illustrate two embodiments of stiffening features that may be used in transition sections 110.

As illustrated in the figures, the stiffening features may be altered at a distal end 518 so that a portion of the stiffening feature will have a reduced stiffness and may be integrated into transition portion 110. For example, FIG. 5A shows a stiffening feature that includes circumferential cuts at distal end 518 to reduce stiffness. Similarly, as shown in the embodiment of FIG. 5B, the stiffening feature may have a reduced cross-sectional area towards distal end 518, resulting in a reduction in stiffness. Preferably, the stiffening feature would not be reduced to a sharp point at its distal end. In addition, when a wire is employed, the diameter of the wire may be reduced over a portion of its length to create the stiffness transition. Preferably, the wire diameter would be reduced from approximately 0.017 inch to 0.006 inch.

In addition, in some cases a clinician may wish to increase the stiffness of the guidewire without exchanging guidewires. Catheter 100 may be used to increase the stiffness of the guidewire while leaving the guidewire in place and the distal tip may further assist in breaching a stenosis. The variable over-the-wire length of catheter 100 simplifies such a procedure.

Catheter 100 can be loaded on guidewire 120 while maintaining control over the guidewire and without requiring wire extensions. First, guide member 114 is slid to the distal end of guideway 112. Distal portion 108 is then loaded onto the proximal end of guidewire 120. Catheter 100 is than advanced until the proximal end of guidewire 120 exits guide member 114. Since guide member 114 provides a clinician with direct control over guidewire 120 at any position along proximal shaft 106, only a short distance of guidewire 120 outside of the body is required to load catheter shaft 102 onto guidewire 120 while still allowing for independent control of both. Guide member 114 and guidewire 120 may then be held in place as catheter 100 is advanced. In this way, control is maintained over guidewire 120 during the entire procedure. After distal tip 122 of catheter 100 reaches the tip of guidewire 120, the combination of guidewire 120 and catheter 100 may be advanced through the blockage.

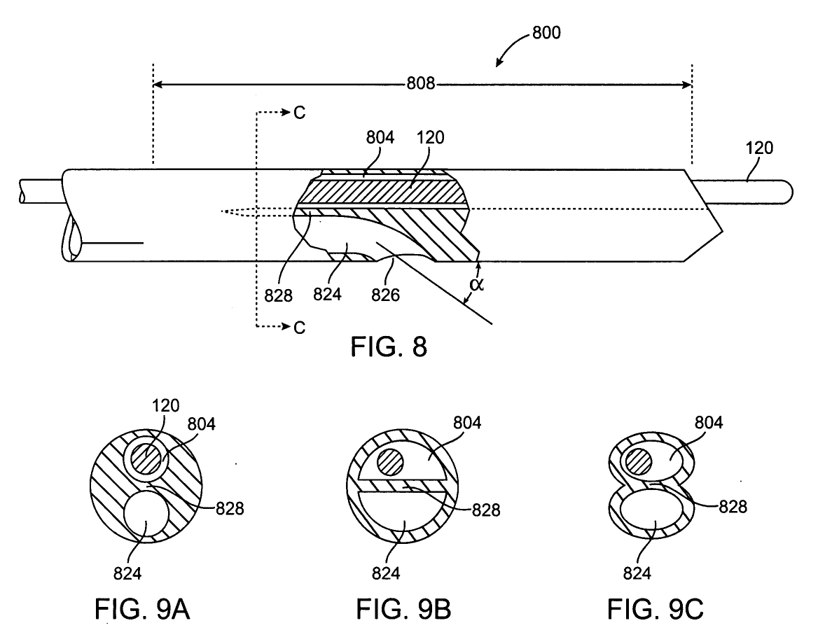

FIG. 8 illustrates an alternative embodiment that includes a branch lumen 824 in a distal portion 808 to aid a clinician in navigating a guidewire through particularly tortuous vessels. The proximal end of branch lumen 824 extends generally parallel to a guidewire lumen 804. Branch lumen 824 curves at a distal end thereof so that it passes through a side wall of catheter 800 at a branch lumen exit 826. Branch lumen 824 intersects the side wall of catheter 800 at an angle a which is greater than 0° and may be as large as 90°. Branch lumen 824 is partitioned from guidewire lumen 804 in distal portion 808 by a branch partition 828.

The cross-sectional shape of distal portion 808 of catheter 800 may vary as shown in FIGS. 9A-C which are cross-sectional views of various embodiments of FIG. 8 taken along line C-C. For example, FIG. 9A illustrates one embodiment of catheter 800 where the cross-section of distal section 808 and both guidewire lumen 804 and branch lumen 824 are circular. FIGS. 9B and 9C illustrate alternative embodiments where guidewire lumen 804 and branch lumen 824 are generally D-shaped or oval. It shall be appreciated that guidewire lumen 804, branch lumen 824 and distal portion 808 may have any cross-sectional shape that will allow a guidewire to be slideably received therein.

As discussed above branch lumen 824 allows guidewire 120 to be guided through a particularly sharp turn that it otherwise would have difficulty being navigated through and which could result in damage to the surrounding tissue if attempted without the use of catheter 800. In one method of using branch lumen 824, catheter 800 may be loaded onto guidewire 120 that is located past the desired path of guidewire 120. Catheter 800 would then be loaded onto guidewire 120 until branch lumen exit 826 is aligned with the desired path. While catheter 800 is held stationary, guidewire 120 would be partially retracted until the distal tip of guidewire 120 is located proximal to branch partition 828. Guidewire 120 is then advanced into branch lumen 824 and out branch lumen exit 826.

Alternatively, a second guidewire may be backloaded into branch lumen 824 of catheter 800. In order to backload the second guidewire into branch lumen 824, the proximal end of the guidewire may be inserted into branch lumen exit 826. The second guidewire is then slid further proximal into branch lumen 824 until the tip of the guidewire is located within branch lumen 824. The combined catheter 800 and backloaded guidewire may then be advanced over an indwelling guidewire until branch lumen exit 826 is aligned with the desired path. Then, the second guidewire is advanced out of branch lumen exit 826 along the desired path and catheter 800 may be removed.

A series of catheters may be provided with branch lumens that exit the distal portion at different angles. During a procedure, a clinician can select the appropriate catheter so that a guidewire may be directed through a particularly tortuous vessel. After a guidewire is inserted, the catheter may be removed and a catheter that is designed to perform a therapeutic procedure may be loaded on the guidewire. As will be described in greater detail below, the catheter is provided with a guide member to simplify guidewire and catheter exchange procedures. The therapeutic catheter may be easily guided on the pre-placed guidewire through the tortuous vessel to the treatment site where the therapy is then performed.

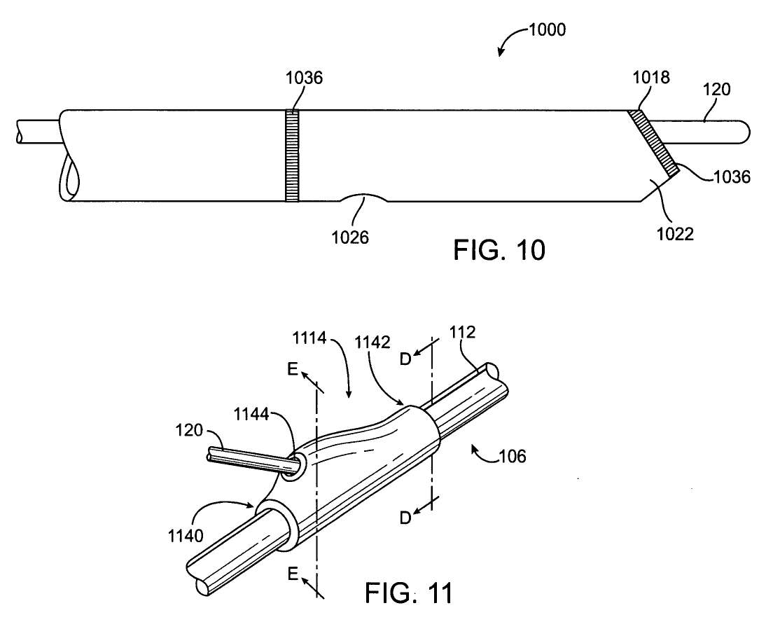

As shown in FIG. 10, radiopaque markers 1036 may be included on a catheter 1000. Radiopaque markers 1036 help a clinician to fluoroscopically view and locate catheter 1000 at a treatment site. Various configurations of radiopaque markers 1036 may be used. For example, radiopaque marker 1036 may be located on a distal tip 1022 so that the location of distal tip 1022 is fluoroscopically viewable. Radiopaque markers 1036 may be located adjacent to a branch lumen exit 1026, as an alternative to or in addition to radiopaque markers on distal tip 1022, so that the branch lumen exit 1026 may be precisely located.

As shown, radiopaque markers 1036 may be radiopaque stripes. Such radiopaque markers may be constructed by encapsulating a radiopaque material, such as a metallic ring, within the material of catheter shaft. Alternatively a portion of the catheter shaft may be made radiopaque for example by constructing the portion from a radiopaque polymer. For example a polymer may be mixed with a radiopaque filler such as barium sulfate, bismuth trioxide, bismuth subcarbonate or tungsten.

Guide member 114 may have one of many forms depending on the required utility. For example, guide member 114 may be used to vary the effective OTW length of catheter 100 in which case guide member 114 provides a proximal exit for guidewire 120. Guide member 114 may alternatively allow direct manipulation of guidewire 120 that is entirely disposed within guidewire lumen 104. In general, guide member 114 allows a clinician to manipulate guidewire 120 independently from catheter shaft 102 during a procedure.

FIGS. 11-13 illustrate one embodiment of a guide member 1114. Guide member 1114 has proximal and distal ends, 1140 and 1142 respectively. A catheter receiving bore 1250 extends longitudinally through guide member 1114 from guide member proximal end 1140 to distal end 1142. Guide member 1114 includes a proximal spreader member 1246 and a distal spreader member 1248 extending radially into catheter receiving bore 1250. The pair of spreader members serve to locally spread open guideway 112 when guide member 1114 is slideably mounted on proximal portion 106. A guidewire passageway 1144 extends through guide member 1114 such that the distalmost end of guidewire passageway 1144 intersects catheter receiving bore 1250 at a shallow angle, preferably ranging from 3° to 15°, at a location between proximal spreader member 1246 and distal spreader member 1248. As distinguished from proximal spreader member 1246, distal spreader member 1248 should not project into guidewire lumen 104, where it could interfere with guidewire 120.

Guide member 1114 may be molded from a rigid plastic material, such as nylon or a nylon based co-polymer, that is preferably lubricous. Alternatively, guide member 1114 may be made of a suitable metal, such as stainless steel, or guide member 1114 may have both metal components and plastic components. For ease in manufacturing, guide member 1114 may be comprised of molded parts that snap-fit together to form the final configuration.

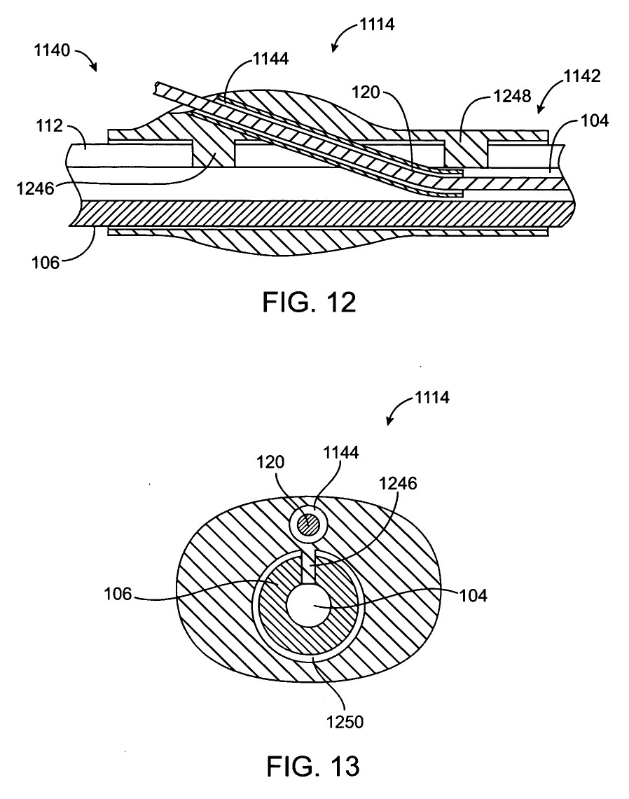

Proximal portion 106 and guidewire 120 both extend through guide member 1114 and merge so that guidewire 120 extends into guidewire lumen 104, as shown in FIG. 12. Proximal portion 106 extends through catheter receiving bore 1250 of guide member 1114, engaging proximal spreader member 1246 therein. Proximal spreader member 1246 extends through guideway 112 in proximal portion 106 to spread guideway 112 apart. Guidewire 120 may extend through guidewire passageway 1144 into catheter receiving bore 1250 and further into guidewire lumen 104 through the spread open guideway 112. As proximal portion 106 is drawn through guide member 1114, the once spread open guideway 112 has a tendency to close due to the choice of materials and configuration of catheter shaft 102, thus enclosing guidewire 120 within guidewire lumen 104.

In an alternative maneuver, guidewire 120 may be inserted or removed through guidewire passageway 1144, while guide member 1114 is held stationary with respect to proximal portion 106. In this fashion, a guidewire exchange may be performed. In yet another procedure, guidewire 120 and proximal portion 106 can be held relatively still while guide member 1114 is translated, thus “unzipping” and “zipping” guidewire 120 and proximal portion 106 transversely apart or together, depending on which direction guide member 1114 is moved.

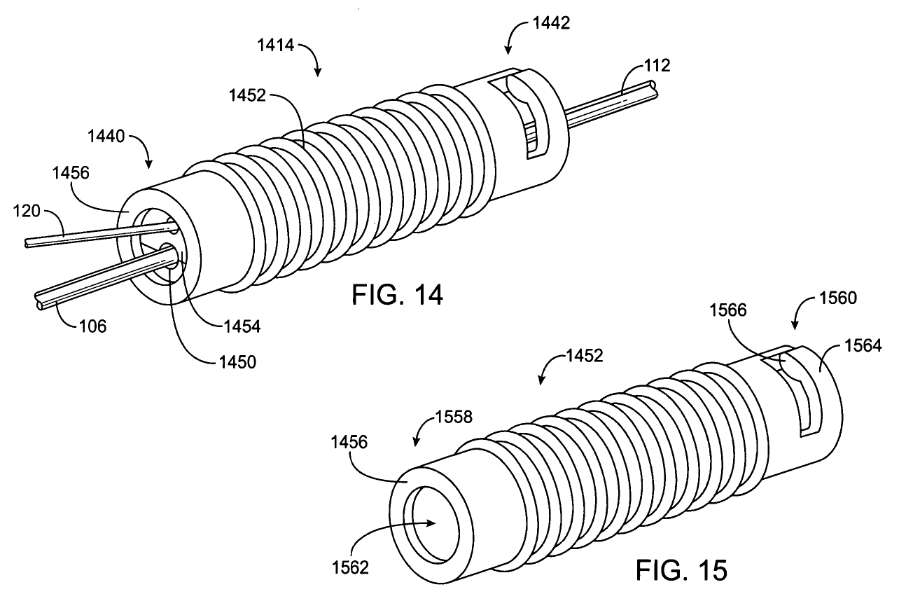

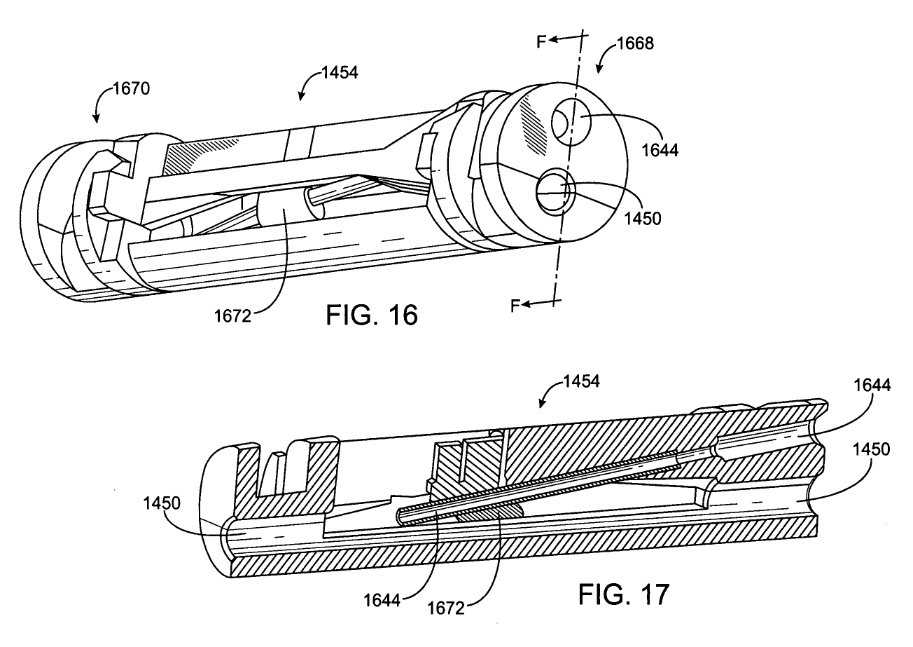

FIGS. 14-17 show an alternate embodiment of a guide member 1414. Guide member 1414 surrounds proximal portion 106 and has a proximal end 1440 and a distal end 1442. Guide member 1414 has an outer tubular member 1452 with proximal and distal ends, 1558 and 1560 respectively, and a longitudinal bore 1562 sized to receive an inner body 1454. The outer tubular member 1452 freely rotates about inner body 1454 but is coupled to inner body 1454 to resist relative axial movement between outer tubular member 1452 and inner body 1454. A stop shoulder 1456, positioned on proximal end 1558 of the outer tubular member 1452, consists of an annular wall that extends radially into longitudinal bore 1562. The stop shoulder 1456 prevents inner body 1454 from slipping out of outer tubular member 1452 through proximal end 1558 of outer tubular member 1452.

Two retaining arms 1564 are disposed on distal end 1560 of outer tubular member 1452. Retaining arms 1564 consist of two arcuate arms that form a portion of outer tubular member 1452. Each arm 1564 contains a tab 1566 that extends into longitudinal bore 1562 of outer tubular member 1452 at its distal end 1560. When guide member 1414 is assembled, tabs 1566 prevent inner body 1454 from slipping out of outer tubular member 1452 through its distal end 1560. Retaining arms 1564 are flexible in the radial direction and may be flexed radially outward. The flexibility allows tabs 1566 to be temporarily removed from the longitudinal bore 1562 to permit insertion and removal of inner body 1454 during the assembly or disassembly of guide member 1414. While two tabs 1566 are shown positioned 180° apart, a different number of tabs may be used, provided they are spaced sufficiently to prevent inner body 1454 from slipping out of outer tubular member 1452. Although the stop shoulder 1456 and retaining arms 1564 are described as integral parts of the outer tubular member, it should be understood that those features may be created by separate elements such as threaded caps.

Inner body 1454, generally functions as guide member 1114, of the previously discussed embodiment. Inner body 1454 has proximal and distal ends, 1668 and 1670 respectively. Catheter receiving bore 1450 extends longitudinally through inner body 1454 from proximal end 1668 to distal end 1670. In the present embodiment, unlike the embodiment shown in FIGS. 11-13, guide member 1414 employs a single keel spreader member 1672. Keel spreader member 1672 serves to locally spread open guideway 112 when guide member 1414 is slideably mounted on proximal portion 106. Guidewire passageway 1644 extends through inner body 1454 such that its distalmost end intersects catheter receiving bore 1450 at a shallow angle, preferably ranging from 3° to 15°. Guidewire passageway 1644 extends through keel spreader member 1672 to assure that guidewire 120 travels unobstructed through the spread guideway 112.

It shall be understood that the single keel design may be substituted for the dual spreader design, shown in FIG. 12, and vice versa. In addition, like guide member 1114, guide member 1414 may be molded from a rigid plastic material, such as nylon or nylon based co-polymers, that is preferably lubricous. Alternatively, guide member 1414 may be made of a suitable metal, such as stainless steel, or guide member 1414 may have both metal components and plastic components. For ease in manufacturing, guide member 1414 may be comprised of molded parts that snap-fit together to form the final configuration.

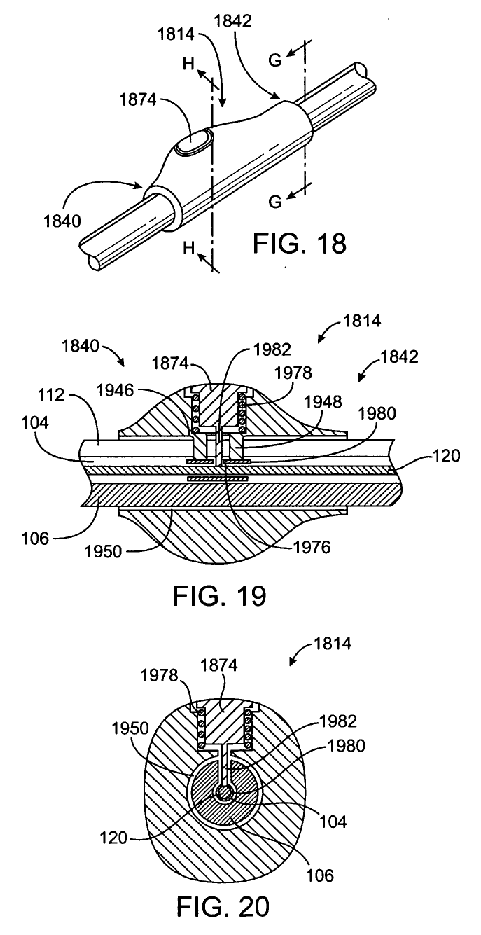

A further alternative embodiment of the guide member is illustrated in FIGS. 18-20. In this embodiment, guide member 1814 provides direct control over axial movement of indwelling guidewire 120. Such a guide member is disclosed in U.S. Patent Application Publication 2004-0039372 A1 published Feb. 26, 2004, the disclosure of which is incorporated by reference in its entirety herein.

As shown in FIG. 19, a guide member 1814 has a main body having both proximal and distal ends, 1840 and 1842 respectively. A catheter receiving bore 1950 extends longitudinally through guide member 1814 from proximal end 1840 to distal end 1842. Guide member 1814 includes a proximal spreader member 1946 and a distal spreader member 1948 extending radially into catheter receiving bore 1950. In addition, a tubular guidewire receiver 1980 is mounted to proximal and distal spreader members, 1946 and 1948 respectively, within catheter receiving bore 1950 and is sized to slideably receive guidewire 120. The pair of spreader members serve to locally spread open guideway 112 and provide a means for holding tubular guidewire receiver 1980 within guidewire lumen 104 when guide member 1814 is slideably mounted on proximal portion 106. Tubular guidewire receiver 1980 has a side opening 1976 sized to receive a clamp member 1982. Proximal spreader member 1946 and distal spreader member 1948 serve to align proximal portion 106 within catheter receiving bore 1950 and especially to align guideway 112 with side opening 1976 on tubular guidewire receiver 1980.

Clamp member 1982 extends radially inward from a clamp control member 1874. Clamp control member 1874 and clamp member 1982 extend through the guide member 1814 and allow a clinician to manually engage a clamping force on guidewire 120. In the present embodiment, a clamp spring 1978 is mounted to clamp control member 1874 and guide member 1814. Clamp spring 1978 holds clamp member 1982 and clamp control member 1874 in a disengaged state when no external force is placed on clamp control member 1874. When clamp control member 1874 is pressed and clamp spring 1978 is compressed, it causes clamp member 1982 to extend further radially into the catheter receiving bore 1950, through side opening 1976 in tubular guidewire receiver 1980 and against guidewire 120. That engagement with guidewire 120 results in a frictional force that resists relative movement between guidewire 120 and guide member 1814 allowing a clinician to directly control the axial location of guidewire 120 within catheter 100.

Like guide members 1114 and 1414, guide member 1814 may be molded from a rigid plastic material, such as nylon or nylon based co-polymers, that is preferably lubricous. Alternatively, guide member 1814 may be made of a suitable metal, such as stainless steel, or guide member 1814 may have both metal components and plastic components. For ease in manufacturing, guide member 1814 may be comprised of molded parts that snap-fit together to form the final configuration.

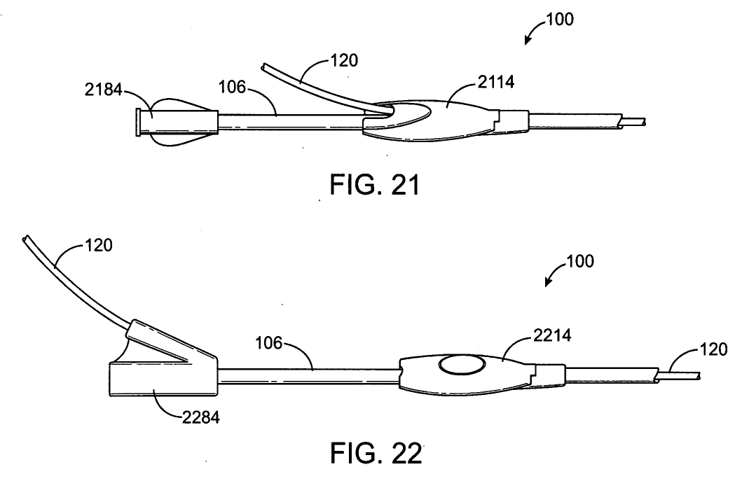

As shown in FIG. 1, the far proximal end 116 of the catheter 100 terminates with a hub 184. Hub 184 may be tailored to the type of guide member employed. As shown in FIG. 21, where a guide member 2114 is one of the types shown in FIGS. 11-17, guide member 2114 provides a proximal exit for guidewire 120 from guidewire lumen 104 and as a result hub 2184 would only require an exit for the lumen of catheter shaft 102 at proximal end 116. On the other hand, for a guide member 2214 of the type shown in FIGS. 18-20 as shown in FIG. 22, a hub 2284 providing a guidewire exit and a catheter shaft lumen exit would be required, such as a Tuohy-Borst fitting.

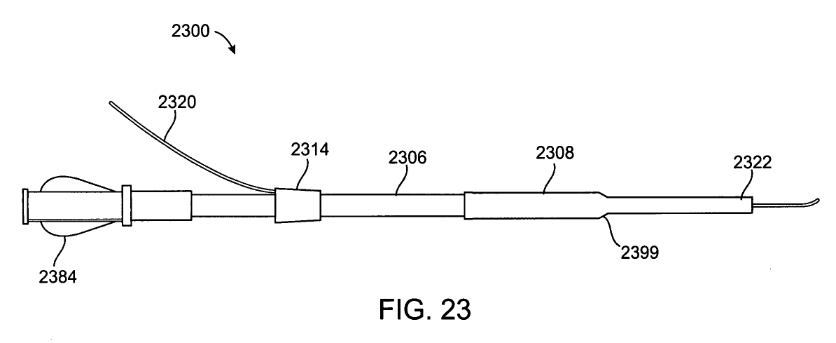

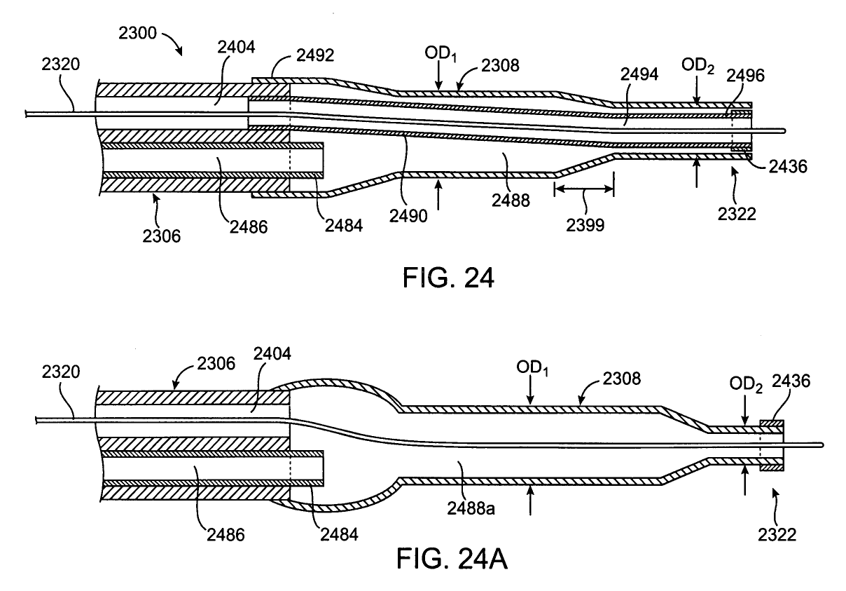

FIGS. 23-25 show another embodiment of a catheter for guidewire placement for use as a microcatheter in treating chronic total, or near total, occlusions. The catheter supports a guidewire in aiding it to cross, for instance, chronic total occlusions. As shown in FIG. 23, catheter 2300 includes a proximal shaft portion 2306 having a hub 2384 attached to its proximal end and a guide member 2314. As in the embodiments shown in FIGS. 1, 11 and 21, guide member 2314 slides longitudinally along proximal shaft portion 2306 and allows a clinician to access a guidewire lumen 2404 through a guideway (not shown). However in this embodiment, proximal shaft portion 2306 is a dual-lumen shaft having guidewire lumen 2404 and an auxiliary lumen 2486, which may be used for dye and/or drug delivery, and/or for taking pressure or other diagnostic measurements. Auxiliary lumen 2486 may be lined by a hypotube 2484, as shown in FIGS. 24 and 24A. Dual-lumen proximal shaft portion 2306 may be of a construction as disclosed in U.S. Pat. Nos. 6,800,065 and 6,893,417, which were previously incorporated by reference.

A distal shaft portion 2308 is attached to a distal end of proximal shaft portion 2306. In the embodiment shown in FIG. 24, distal portion 2308 includes an inner shaft 2490 that has a distal guidewire lumen 2494 for extending guidewire lumen 2404 of proximal shaft portion 2306 to a distal tip 2322 of catheter 2300. Within distal shaft portion 2308, an outer lumen 2488 encircles inner shaft 2490 for communicating a dye, drug or diagnostic instrument delivered through auxiliary lumen 2486 to the catheter's distal tip 2322. In a further embodiment, a distal end 2496 of inner shaft 2490 may be tack bonded to an inner surface of distal shaft portion 2308 at or proximate to distal tip 2322.

In the embodiment shown in FIG. 24A, distal shaft portion 2308 does not include inner shaft 2490, but instead provides a single distal lumen 2488a for delivering the guidewire and drug or dye, if any is used, through distal shaft portion 2308 to the catheter's distal tip 2322. Alternatively, distal portion 2308 may include an inner tube (not shown) attached to the distal end of hypotube 2484 for delivering drug or dye to the catheter's distal tip 2322, to thereby prevent any delivered substance from entering proximal guidewire lumen 2404. In each of the embodiments shown in FIGS. 24 and 24A, a distal end of either distal inner shaft 2490 or distal shaft portion 2308 may be surrounded by a radiopaque marker band 2436 to aid in fluoroscopic observation during manipulation of catheter 2300 through a patient's vasculature.

Distal shaft portion 2308 includes a proximal end 2492 that is stretched to surround the distal end of proximal portion 2306 to be bonded thereto. Distal shaft proximal end 2492 may be spot welded, laser welded or secured using a bonding sleeve or adhesive to proximal shaft portion 2308, as would be apparent to one skilled in the relevant art. Distal shaft portion 2308 includes a necked portion 2399 that provides a transition from a proximal outer diameter, OD₁, to a reduced, distal outer diameter, OD₂, that enables catheter 2300 to have a significantly reduced distal profile. The outer diameter of distal shaft 2308 may range in size from 2 F to 5 F. In one embodiment, distal shaft portion 2308 has an OD₁, of 2.7 F and an OD₂ of 2.5 F allowing catheter 2300 to fit within tightly stenosed and/or totally occluded areas of the vasculature.

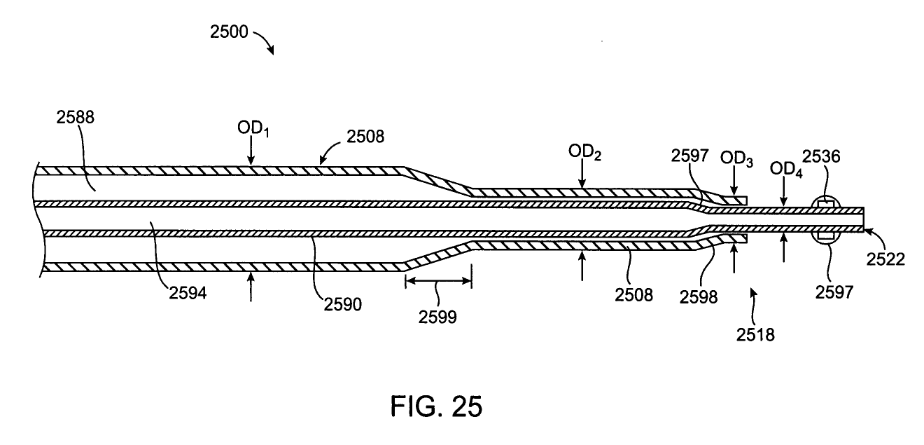

FIG. 25 shows an alternate embodiment of a distal shaft portion 2580 of a catheter 2500 in accordance with the present invention. A proximal portion of catheter 2500 may be similar to any of the foregoing embodiments, and distal portion 2580 is similar to distal portion 2308 shown in FIG. 24, except as noted herein. In this embodiment, distal shaft portion 2508 includes a proximal necked portion 2599 and a distal necked portion 2598. Proximal necked portion 2599 provides a transition from a proximal outer diameter, OD₁, to a first-reduced, distal outer diameter, OD₂. Distal necked portion 2598 provides a further transition from first-reduced, distal outer diameter, OD₂, to a second-reduced, distal outer diameter, OD₃. In addition, inner shaft 2590 that encloses distal guidewire lumen 2594 includes an inner shaft necked portion 2597 to provide a reduced outer diameter, OD₄, as it exits and extends from distal end 2518 of distal shaft portion 2508. In an exemplary embodiment, OD₁, is 2.6 F, OD₂ is 2.3 F, OD₃ is 2.0 F, and OD₄ is 1.6 F. In this manner, di tip 2522 of catheter 2300 has a significantly reduced distal profile making it easier to cross/penetrate chronic totally occluded vessels. The outer diameters, i.e., OD₁, OD₂, and OD₃, of distal shaft 2508 may range in size from 1.8 F to 3.0 F, and the minimum outer diameter, ie., OD₄, of inner shaft 2590 may range in size from 1.3 F to 2.0 F.

As shown in FIG. 25, distal tip 2522 of distal inner shaft 2590 is surrounded by a radiopaque marker band 2536 that is held in place by a heat-shrinkable sheath 2597. Marker band 2536 aids in fluoroscopic observation of catheter 2500 during manipulation of the catheter through a patient's vasculature. In a further embodiment, inner shaft 2590 may be tack bonded to an inner surface of distal shaft portion 2508 at or proximate to its distal end 2518.

In the embodiments of the present invention shown in FIGS. 23-25, distal shaft portions 2308, 2508 and inner shafts 2490, 2590 may be made of polyethylene, PEBAX, nylon, polyurethane, or a co-extrusion or copolymer of these materials. In one embodiment, proximal shaft portion 2306 is comprised of polyethylene and distal shaft portion 2308 is comprised of an inner layer of polyethylene and an outer layer of PEBAX to facilitate bonding of distal shaft portion 2308 to proximal shaft portion 2306. In a further embodiment, inner shaft 2490 is comprised of an inner layer of PEBAX and an outer layer of polyethylene to facilitate bonding of inner shaft 2490 within guidewire lumen 2402 of proximal shaft 2306.

While this invention has been particularly shown and described with reference to preferred embodiments thereof, it will be understood by those skilled in the art that various changes in form and details may be made therein without departing from the spirit and scope of the invention.

What is claimed is:

1. A catheter for guidewire placement comprising:
a tubular catheter shaft having a proximal portion, a distal portion, and a distal tip;
a guidewire lumen extending longitudinally through the catheter shaft;
a guideway extending longitudinally along the length of the proximal portion and radially from the guidewire lumen to an outer surface of the proximal portion;
a guide member slideably coupled to the catheter shaft that is configured to provide access to the guidewire lumen via the longitudinal guideway; and
a branch lumen extending through the distal portion and exiting through a side wall of the catheter shaft at a branch lumen exit.

2. The catheter of claim 1, wherein a material of the distal tip is harder than a material of the distal portion of the catheter shaft.

3. The catheter of claim 1, wherein the distal tip includes a reinforcing member.

4. The catheter of claim 3, wherein the reinforcing member is made of a radiopaque material.

5. The catheter of claim 1, further comprising:
at least one stiffening member disposed within a wall of the catheter shaft.

6. The catheter of claim 1, further comprising:
at least one stiffening member disposed within the guidewire lumen.

7. The catheter system of claim 1, wherein a radiopaque marker is disposed adjacent to the branch lumen exit.

8. The catheter of claim 1, wherein the branch lumen exits through the side wall of the catheter shaft at an angle a that is greater than 0° and less than 90° with respect to the side wall of the catheter shaft.

9. A catheter system comprising:
a tubular catheter shaft having a proximal portion, a distal portion, and a distal tip, wherein the distal tip is of a harder material than the distal portion;
a guidewire lumen extending longitudinally through the catheter shaft;
a guideway extending longitudinally along the length of the proximal portion and radially from the guidewire lumen to an outer surface of the proximal portion; and
a guide member slideably coupled to the catheter shaft that is configured to provide access to the guidewire lumen via the guideway.

10. The catheter of claim 9, wherein the distal tip includes a reinforcing member.

11. The catheter of claim 10, wherein the reinforcing member is made of a radiopaque material.

12. The catheter system of claim 9, further comprising:
a branch lumen extending through the distal section and exiting through a side wall of the catheter shaft at a branch lumen exit.

13. The catheter of claim 9, further comprising:
at least one stiffening member disposed within a wall of the catheter shaft.

14. A catheter system comprising:
a catheter shaft having having a distal tip;
a guidewire lumen extending longitudinally through the catheter shaft, the guidewire lumen having a diameter that reduces from a first diameter to a second diameter proximate to the distal tip;
a guideway extending longitudinally along a length of the catheter shaft and radially from the guidewire lumen to an outer surface of the catheter shaft; and
a guide member slideably coupled to the catheter shaft for accessing the guidewire lumen via the guideway.

15. The catheter of claim 14, wherein the distal tip includes a reinforcing member.

16. The catheter system of claim 14, further comprising:
a branch lumen extending through a distal section of the catheter shaft and exiting through a side wall of the catheter shaft at a branch lumen exit.

17. The catheter of claim 14, further comprising:
at least one stiffening member disposed within a wall of the catheter shaft.

18. A catheter for guidewire placement comprising:
a catheter shaft having a proximal portion and a distal portion, the distal shaft portion including a necked region that transitions the distal shaft from a first outer diameter to a reduced second outer diameter;
a guidewire lumen and an auxiliary lumen extending longitudinally through the catheter shaft in a side-by-side arrangement in the proximal shaft, wherein the auxiliary lumen accommodates drug or dye infusion;
a guideway extending longitudinally along the length of the proximal portion and radially from the guidewire lumen to an outer surface of the proximal portion; and
a guide member slideably coupled to the catheter shaft that is configured to provide access to the guidewire lumen via the longitudinal guideway.

19. The catheter of claim 18, wherein the first outer diameter of the distal shaft portion is 2.7 F and the second outer diameter is 2.5 F to fit within tightly stenosed occlusions.

20. The catheter of claim 18, wherein the distal shaft portion further includes a distal necked region that transitions the distal shaft to a third outer diameter that is less than the second outer diameter.

21. The catheter of claim 20, wherein the distal shaft portion includes an inner tubular member that extends the proximal shaft guidewire lumen to a distal tip of the catheter shaft, such that the guidewire lumen is in a substantially coaxial arrangement with the auxiliary lumen within the distal shaft.

22. The catheter of claim 21, wherein the inner tubular member includes a necked region proximal to the distal necked region of the distal shaft portion that transitions the inner tubular member from a first outer diameter to a reduced second outer diameter.

23. The catheter of claim 22, wherein a distal end of the inner tubular member is the distal tip of the catheter shaft that extends distally of a distal end of the distal shaft portion.

24. The catheter of claim 18, wherein the distal shaft portion includes an inner tubular member that extends the proximal shaft guidewire lumen to a distal tip of the catheter shaft, such that the guidewire lumen is in a substantially coaxial arrangement with the auxiliary lumen within the distal shaft.

25. The catheter of claim 18, wherein the distal shaft portion includes an inner tubular member that extends the proximal shaft auxiliary lumen to a distal tip of the catheter shaft, such that the auxiliary lumen is in a substantially coaxial arrangement with the guidewire lumen within the distal shaft.

26. A method of using a catheter, comprising the steps of:
providing a catheter comprising a tubular catheter shaft having a proximal portion, a transition portion, a distal portion, and a distal tip; a guidewire lumen extending longitudinally through the catheter shaft; a guideway extending longitudinally along the length of the proximal portion and radially from the guidewire lumen to an outer surface of the proximal portion; a guide member slideably coupled to the catheter shaft for accessing the guidewire lumen via the guideway; and a branch lumen extending through the distal portion and exiting through a side wall of the catheter shaft at a branch lumen exit;
providing a guidewire;
backloading the guidewire into the catheter by inserting a proximal end of the guidewire into the branch lumen exit and sliding the guidewire further proximal into the branch lumen until the guidewire is fully inserted into the branch lumen;
advancing the catheter so the branch lumen exit is aligned with a tortuous vessel; and
advancing the guidewire distally through the branch lumen so that a distal tip of the guidewire exits the branch lumen exit and enters the tortuous vessel.
